(19) United States
(12) Patent Application Publication
(10) Pub. No.: US 2019/0111100 A1
(43) Pub. Date: Apr. 18, 2019

(54) USES OF CONVALLARIA ESSENTIAL OIL

(71) Applicant: TCI Co., Ltd., Taipei (TW)

(72) Inventor: Yung-Hsiang LIN, Taipei (TW)

(21) Appl. No.: 16/158,619

(22) Filed: Oct. 12, 2018

Related U.S. Application Data
(60) Provisional application No. 62/573,929, filed on Oct. 18, 2017.

(30) Foreign Application Priority Data
Sep. 21, 2018 (TW) 107133373

(51) Int. Cl. A61K 36/88; A61P 9/04; A61P 37/00

(52) U.S. Cl. CPC A61K 36/88; A61K 9/0073; A61P 37/00; A61P 9/04

(57) ABSTRACT

A method for at least one of stimulating natural killer cells (NK cells), enhancing effects of T helper 1 cells (Th1 cells), and inhibiting differentiation of CD4⁺ T cell-dependent macrophages is provided, wherein the method comprises administering to a subject in need an effective amount of convallaria essential oil.

### CROSS REFERENCE TO RELATED APPLICATIONS

This application claims priority to U.S. Provisional Application Ser. No. 62/573,929 filed on Oct. 18, 2017, in the United States Patent and Trademark Office, and to Taiwan Patent Application No. 107133373 filed on Sep. 21, 2018, in the Taiwan Intellectual Property Office, the disclosures of which are incorporated herein in their entirety by reference.

### FIELD OF THE INVENTION

The present invention relates to the use of convallaria essential oil, including the use of convallaria essential oil in increasing expression of IL-12A gene. The present invention also relates to the use of convallaria essential oil in stimulating natural killer cells, enhancing effects of T helper 1 cells, and/or inhibiting differentiation of CD4⁺ T cell-dependent macrophages, so as to provide the following effects: enhancing immunity, inhibiting cardiac fibrosis, and inhibiting cardiac fibrosis-induced diseases.

### BACKGROUND OF THE INVENTION

The immune system is a host defense system and plays an important role in maintaining the health of organisms. Depending on the specificity and memory to antigen, the immune system could be classified into the innate immunity and the adaptive immunity. Under normal circumstances, immunocytes can serve appropriate immune functions, such as helping with the defense against external pathogens (e.g., bacteria, fungi, viruses, microorganisms, and various antigens) as well as killing the mal-functional cells (e.g., aged cells, gene-mutated cells, pathogen-infected cells or cancer cells) within the organisms. Therefore, an enhancement of immunity is beneficial to the maintenance of the health of organisms.

In current medical research, though it is known that appropriate exercise and diet as well as sufficient sleep can enhance the immunity of a subject, modern people are generally deficient in exercise and sleep and usually have a nutritional imbalanced diet. Thus, it becomes impractical to achieve the goal of strengthening the immune system through exercise, sleep and diet. Therefore, for busy modern people, it is necessary to develop an alternative approach that can be adopted easily without affecting their daily life.

Inventors of the present invention incidentally discovered that convallaria essential oil can increase the expression of IL-12A gene, and thus, is effective in stimulating natural killer cells, enhancing effects of T helper 1 cells, and inhibiting differentiation of CD4⁺ T cell-dependent macrophages. Hence, convallaria essential oil can be used for enhancing immunity, inhibiting cardiac fibrosis, inhibiting cardiac fibrosis-induced heart failure and/or inhibiting cardiac fibrosis-induced ventricular stiffness.

### SUMMARY OF THE INVENTION

An objective of the present invention is to provide a use of convallaria essential oil in manufacturing of a pharmaceutical composition for at least one of stimulating natural killer cells, enhancing effects of T helper 1 cells, and inhibiting differentiation of CD4⁺ T cell-dependent macrophages. Preferably, the pharmaceutical composition is for at least one of enhancing immunity, inhibiting cardiac fibrosis, inhibiting cardiac fibrosis-induced heart failure, and inhibiting cardiac fibrosis-induced ventricular stiffness. Preferably, the pharmaceutical composition is provided in a form for inhalation administration or transdermal administration.

Another objective of the present invention is to provide a use of convallaria essential oil in the manufacture of a pharmaceutical composition for increasing expression of IL-12A gene. Preferably, the pharmaceutical composition is provided in a form for inhalation administration or transdermal administration.

Still another objective of the present invention is to provide a pharmaceutical composition for stimulating natural killer cells (NK cells), enhancing effects of T helper 1 cells (Th1 cells), and/or inhibiting differentiation of CD4⁺ T cell-dependent macrophages. The pharmaceutical composition comprises an effective amount of convallaria essential oil. Preferably, the pharmaceutical composition is for enhancing immunity, inhibiting cardiac fibrosis, inhibiting cardiac fibrosis-induced heart failure, and/or inhibiting cardiac fibrosis-induced ventricular stiffness. Preferably, the pharmaceutical composition is provided in a form for inhalation administration or transdermal administration.

Yet another objective of the present invention is to provide a pharmaceutical composition for increasing expression of IL-12A gene. The pharmaceutical composition comprises an effective amount of convallaria essential oil. Preferably, the pharmaceutical composition is provided in a form for inhalation administration or transdermal administration.

Yet another objective of the present invention is to provide a method for at least one of stimulating natural killer cells (NK cells), enhancing effects of T helper 1 cells (Th1 cells), and inhibiting differentiation of CD4⁺ T cell-dependent macrophages, comprising administering to a subject in need an effective amount of convallaria essential oil. In the method of the present invention, the convallaria essential oil can be administered to the subject as the pharmaceutical composition described above. Preferably, the method of the present invention is for at least one of enhancing immunity, inhibiting cardiac fibrosis, inhibiting cardiac fibrosis-induced heart failure, and inhibiting cardiac fibrosis-induced ventricular stiffness.

Yet another objective of the present invention is to provide a method for increasing expression of IL-12A gene, comprising administering to a subject in need an effective amount of convallaria essential oil. In the method of the present invention, the convallaria essential oil can be administered to the subject as the pharmaceutical composition described above.

The detailed technology and preferred embodiments implemented for the present invention will be described in the following paragraphs accompanying the appended drawings for persons skilled in the field to well appreciate the features of the claimed invention.

### BRIEF DESCRIPTION OF THE DRAWINGS

FIG. 1 shows the influence of the convallaria essential oil on the expression of IL-12A gene in peripheral blood mononuclear cells (PBMCs), wherein the result of “W0” group refers to the expression level before the administration of convallaria essential oil, and the results of “W1” group and “W2” group refer to the expression levels after the continuous administration of convallaria essential oil for one week and two weeks, respectively (* represents the result is significantly different from that of the “W0” group, p<0.05).

### DESCRIPTION OF THE PREFERRED EMBODIMENT

The following paragraphs will describe some of the embodiments of the present invention in detail. However, without departing from the spirit of the present invention, the present invention may be embodied in various embodiments and should not be limited to the embodiments described in the specification.

Unless otherwise indicated herein, the expression “a,” “an,” “the,” or the like recited in the specification of the present invention (especially in the claims) are intended to include both the singular and plural forms. The terms “enhance immunity”, “enhancing immunity”, and “enhancement of immunity” recited in the specification refer to enhancing the abilities of immunocytes in a subject to defense against pathogens (e.g., bacteria, virus, etc.) or to recognize the mal-functional cells (e.g., gene-mutated cells, cancer cells, etc.). The terms “inhibit disease(s)”, “inhibiting disease(s)” and “inhibition of disease(s)” recited in the specification refer to preventing or delaying the onset and deterioration of a disease (e.g., cardiac fibrosis) or the symptoms related to the disease in a subject, so as to maintain the progression of diseases in a steady state or lead the subject to a healthy state. The term “subject” recited in the specification refers to human or non-human animals (e.g., dogs and cats).

Research results have shown that an increment in the expression level of IL-12A gene may stimulate natural killer cells (NK cells) and enhance effects of T helper 1 cells (Th1 cells), thereby enhancing immunity, and these can be noted in “A Controversial Role for IL-12 in Immune Response and Bone Resorption at Apical Periodontal Site. Clinical and Developmental Immunology. 2010; 2010:327417”, which is entirely incorporated hereinto by reference. Furthermore, research results have also shown that a deficiency of IL-12A gene may promote the differentiation of CD4⁺ T cell-dependent macrophages and enhance cardiac fibrosis. These can be noted in “Interleukin-12p35 deletion promotes CD4 T-cell-dependent macrophage differentiation and enhances angiotensin II-Induced cardiac fibrosis. Arterioscler Thromb Vasc Biol. 2012 July; 32(7):1662-74”, which is entirely incorporated hereinto by reference.

Therefore, if the expression of IL-12A gene can be increased effectively, the following effects could be provided: stimulating natural killer cells, enhancing effects of T helper 1 cells and inhibiting differentiation of CD4⁺ T cell-dependent macrophages, so that can enhance immunity, inhibit cardiac fibrosis, and/or inhibit cardiac fibrosis-induced diseases.

Essential oil is an oil product containing aromatic compound(s) and is extracted from plants. Specifically, essential oil can be provided by extracting the flowers, leaves, roots, seeds, peels, fruits and/or stems of plant(s) by such as distillation, expeller press, solvent extraction, enfleurage, and supercritical fluid extraction (SFE). Depending on the species and parts of plant(s) from which the essential oil is produced, the essential oil may have different effects and can be used in different forms in daily life or in the pharmaceutical industry as well.

Inventors of the present invention incidentally discovered that convallaria essential oil can effectively increase expression of IL-12A gene. Therefore, the present invention relates to uses of the convallaria essential oil, including: using convallaria essential oil in manufacturing a pharmaceutical composition, providing a pharmaceutical composition comprising an effective amount of convallaria essential oil, and providing a method for administering to a subject in need an effective amount of the aforementioned pharmaceutical composition. The pharmaceutical composition provided in accordance with the present invention is for at least one of stimulating natural killer cells, enhancing effects of T helper 1 cells and inhibiting differentiation of CD4⁺ T cell-dependent macrophages. Preferably, the pharmaceutical composition provided in accordance with the present invention is for at least one of enhancing immunity, inhibiting cardiac fibrosis, inhibiting cardiac fibrosis-induced heart failure, and inhibiting cardiac fibrosis-induced ventricular stiffness. Furthermore, the pharmaceutical composition and method provided in accordance with the present invention are also for increasing expression of IL-12A gene.

The convallaria essential oil adopted by the present invention could be obtained from any suitable source. For example, the convallaria essential oil could be obtained by extracting convallaria (e.g., extracting the followers of convallaria via solvent extraction), or could be purchased from the market.

The pharmaceutical composition provided in accordance with the present invention could be administered to a subject in need systemically or topically, and could be delivered by various drug delivery systems (DDSs), such as transdermal drug delivery system, inhalation drug delivery system, etc. For example, to enhance bioavailability, control drug release speed, target the lesion precisely and reduce side effects, the pharmaceutical composition could be delivered by a liposome, a microcapsule, nanoparticles, or microneedles, but is not limited thereby.

Depending on the desired purpose(s), the pharmaceutical composition of the present invention could be provided in any suitable form without particular limitations. For example, the pharmaceutical composition could be provided in a form for inhalation administration, or transdermal administration, but is not limited thereby. Depending on the form and purpose(s), a suitable carrier could be chosen and used to provide the pharmaceutical composition. Examples of the carrier include excipients, diluents, auxiliaries, stabilizers, absorption enhancers, disintegrating agent, hydrotropic agents, emulsifiers, antioxidants, adhesives, binders, tackifiers, dispersants, suspending agents, lubricants, hygroscopic agents, etc.

As a form for inhalation administration, the pharmaceutical composition of the present invention could be optionally aerosolized by any suitable approach to facilitate the entry of the pharmaceutical composition into the respiratory tract. For example, the pharmaceutical composition could be administered through a nebulizer, an aroma burner, an evaporative diffuser, an aroma lamp, an aroma diffuser, an essential oil candle, or an essential oil necklace, but is not limited thereby.

As a form suitable for transdermal administration, the pharmaceutical composition of the present invention could also comprise any pharmaceutically acceptable carrier that will not adversely affect the desired effects of the active ingredient (i.e., convallaria essential oil). Examples of suitable carriers include, but are not limited to, water, mineral oil, propylene glycol, polyethylene oxide, liquid petrolatum, sorbitan monosterate, and polysorbate 60. The pharmaceutical composition could be provided by any suitable method in any suitable form for transdermal administration, such as in the form of an emulsion (such as a massage emulsion), a cream (such as a massage cream), an oil (such as a massage oil), a gel (such as a hydrogel), a paste (such as a dispersing paste, an ointment), a lotion, a spray, or a patch (such as a microneedle patch), etc., but is not limited thereby.

Optionally, the pharmaceutical composition provided in accordance with the present invention could further comprise a suitable amount of additives, such as a flavoring agent, a toner, or a coloring agent for enhancing the palatability and the visual perception of the pharmaceutical composition, and/or a buffer, a conservative, a preservative, an antibacterial agent, or an antifungal agent for improving the stability and storability of the pharmaceutical composition. Optionally, the pharmaceutical composition could further comprise one or more other active ingredient(s) (e.g., Angiotensin Converting Enzyme Inhibitor (ACEI)) to further enhance the effects of the pharmaceutical composition, or to increase the application flexibility and adaptability of the preparation thus provided, as long as the other active ingredients will not adversely affect the desired effects of the active ingredients of the present invention (i.e., convallaria essential oil).

Depending on the needs, age, body weight, and health conditions of the subject as well as the purpose(s), the pharmaceutical composition provided in accordance with the present invention could be dosed at various frequencies, such as once a day, multiple times a day, or once every few days, etc. In addition, the concentration of convallaria essential oil in the pharmaceutical composition could be adjusted depending on the requirements of practical application.

As described above, the present invention also provides a method for at least one of stimulating natural killer cells, enhancing effects of T helper 1 cells, and inhibiting differentiation of CD4⁺ T cell-dependent macrophages, comprising administering to a subject in need an effective amount of convallaria essential oil, wherein the term “a subject in need” refers to a subject in which the number and/or activity of natural killer cells is deficient, the T helper 1 cells are not efficient, and/or the CD4⁺ T cell-dependent macrophages are over differentiated. The present invention also relates to a method for increasing expression of IL-12A gene, comprising administering to a subject in need an effective amount of convallaria essential oil, wherein the term “a subject in need” refers to a subject in which the IL-12A gene is deleted, mutated, or low-expressed. In the method in accordance with the present invention, the convallaria essential oil could be administered to the subject in need as a pharmaceutical composition and the administration type, administration route, administration form, administration frequency, and uses of the pharmaceutical composition are all in line with the above descriptions.

The present invention will be further illustrated in detail with specific examples as follows. However, the following examples are provided only for illustrating the present invention and the scope of the present invention is not limited thereby. The scope of the present invention will be indicated in the appended claims.

### EXAMPLES

### Example 1: Effects of Convallaria Essential Oil on Increasing Expression of IL-12A Gene

### (1-1) Collection of Blood Sample

The following two-week trial was conducted on six persons (six subjects) to understand the effects of convallaria essential oil on human body. During the trail, each subject wore an essential oil necklace containing convallaria essential oil (purchased from Taiwan Specific Plant Essential Oil) for 8 hours every day, and the essential oil necklace was added with 20 μL of convallaria essential oil at nine o'clock every morning to ensure that the amount of essential oil in the necklace was sufficient. And, 6 mL blood sample was collected from each subject prior to the trial (i.e., prior to starting wearing the essential oil necklace containing convallaria essential oil; hereinafter referred to as “W0” group), after starting the trial for one week (i.e., wearing the essential oil necklace containing convallaria essential oil eight hours a day and for one week; hereinafter referred to as “W1” group), and after starting the trial for two weeks (i.e., wearing the essential oil necklace containing convallaria essential oil eight hours a day and for two weeks; hereinafter referred to as “W2” group), respectively, by using VACUETTE® blood collection tubes that contained ethylenediaminetetraacetic acid (EDTA) (product number: 456036). The blood samples thus obtained were used for the following experiments and analysis.

### (1-2) Isolation of Peripheral Blood Mononuclear Cell (PBMC)

The blood samples of “W0” group, “W1” group and “W2” group obtained from Example (1-1) were individually subjected to the following steps to isolate PBMCs from the blood samples of each group:

1. Injecting each blood sample to a 15 mL centrifuge tube, and subjecting the centrifuge tube to a centrifugation at 300 g for 15 minutes (the centrifugation was stopped by natural deceleration);
2. Removing the upper blood plasma layer, taking out 2 mL of buffy coat from the remnant and putting the same to another 15 mL centrifuge tube, and then adding thereinto 2 mL of phosphate buffered saline (PBS) and mixing evenly to provide a diluted buffy coat;
3. Tilting another 15 mL centrifuge tube loaded with 3 mL of Ficoll-Plague Plus (purchased from Sigma, product number: GE17-1440-03) for about 45 degrees, then slowly adding the diluted buffy coat provided by step 2 into the centrifuge tube and carefully returning the centrifuge tube back to the vertical situation, so that the buffy coat and Ficoll-Plague Plus were layered (not mixed), and then, subjecting the centrifuge tube to a centrifugation at 400 g for 40 minutes (the centrifugation was stopped by natural deceleration);
4. Removing the upper liquid layer, taking out 2 mL to 3 mL of the middle layer (i.e., mononuclear cell layer) and putting the same to a new 15 mL centrifuge tube, then, washing the cells with PBS three to five times, and then, subjecting the washed cells to a centrifugation at 300 g for 10 minutes; and
5. Removing the remaining PBS to provide the precipitate, i.e., the PBMCs of “W0” group, “W1” group, or “W2” group.

### (1-3) Gene Expression Analysis

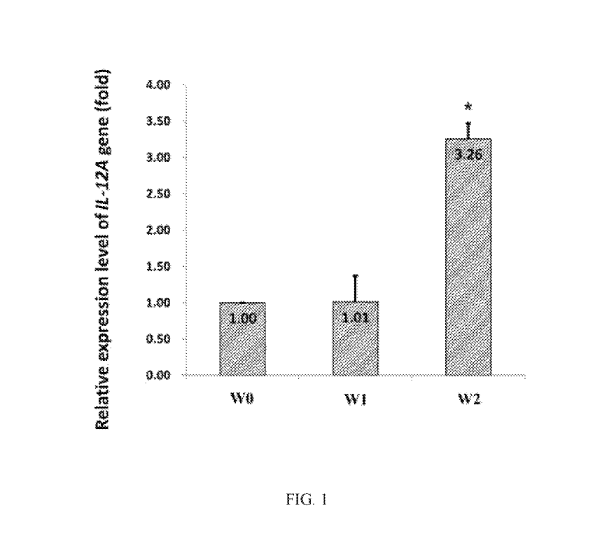

The PBMCs of “W0” group, “W1” group, and “W2” group obtained from Example (1-2) were lysed by an RNA lysis buffer (RB buffer; purchased from Geneaid company). Thereafter, the PBMC lysate of each group was subjected to an RNA extraction with an RNA extraction kit (purchased from Geneaid company), and then the RNA was transcribed into cDNA with a reverse transcriptase (SuperScript® III Reverse Transcriptase; purchased from Invitrogen company). Then, the cDNA of each group was subjected to a quantitative polymerase chain reaction (qPCR) by using an ABI Step One Plus system and a KAPA SYBR FAST qPCR kit to determine the expression level of IL-12A gene in the cells of each group. Finally, the data was analyzed by T.TEST of Excel (one tailed Student's t-test), and the analysis result of “W0” group was used as a basis (i.e., the gene expression of “W0” group was set as 1-fold) to calculate the relative gene expression level of “W1” group and that of “W2” group. The results are shown in FIG. 1.

As shown in FIG. 1, as compared to “W0” group, the expression level of IL-12A gene in the cells of “W2” group significantly increased. These results indicate that convallaria essential oil can effectively increase the expression of IL-12A gene, and thus, is effective in stimulating natural killer cells, enhancing effects of T helper 1 cells and inhibiting differentiation of CD4⁺ T cell-dependent macrophages. Hence, convallaria essential oil can be used for enhancing immunity, inhibiting cardiac fibrosis, inhibiting cardiac fibrosis-induced heart failure, and/or inhibiting cardiac fibrosis-induced ventricular stiffness.

What is claimed is:

1. A method for at least one of stimulating natural killer cells (NK cells), enhancing effects of T helper 1 cells (Th1 cells), and inhibiting differentiation of CD4⁺ T cell-dependent macrophages, comprising administering to a subject in need an effective amount of convallaria essential oil.

2. The method as claimed in claim 1, which is for enhancing immunity.

3. The method as claimed in claim 1, which is for inhibiting cardiac fibrosis.

4. The method as claimed in claim 1, which is for inhibiting cardiac fibrosis-induced heart failure.

5. The method as claimed in claim 1, which is for inhibiting cardiac fibrosis-induced ventricular stiffness.

6. The method as claimed in claim 1, wherein the convallaria essential oil is administered to the subject by at least one of inhalation administration and transdermal administration.

7. The method as claimed in claim 2, wherein the convallaria essential oil is administered to the subject by at least one of inhalation administration and transdermal administration.

8. The method as claimed in claim 3, wherein the convallaria essential oil is administered to the subject by at least one of inhalation administration and transdermal administration.

9. The method as claimed in claim 4, wherein the convallaria essential oil is administered to the subject by at least one of inhalation administration and transdermal administration.

10. The method as claimed in claim 5, wherein the convallaria essential oil is administered to the subject by at least one of inhalation administration and transdermal administration.

11. A method for increasing expression of IL-12A gene, comprising administering to a subject in need an effective amount of convallaria essential oil.

12. The method as claimed in claim 11, wherein the convallaria essential oil is administered to the subject by at least one of inhalation administration and transdermal administration.
